(19) United States
(12) Patent Application Publication
(10) Pub. No.: US 2015/0030552 A1
(43) Pub. Date: Jan. 29, 2015

(54) COSMETIC COMPOSITION

(71) Applicant: Climax Holdings Pty Limited, Thornleigh New South Wales (AU)

(72) Inventor: Wendell Wait, New South Wales (AU)

(73) Assignee: Climax Holdings Pty Limited, Thornleigh New South Wales (AU)

(21) Appl. No.: 14/384,762

(22) PCT Filed: Mar. 13, 2013
(86) PCT No.: PCT/AU2013/000243

§ 371 Date: Sep. 12, 2014

(30) Foreign Application Priority Data
Mar. 13, 2012 (AU) 2012900997

(51) Int. Cl. A61K 8/92; A61K 8/97; A61K 8/891; A61K 8/365; A61K 8/06; A61K 8/44; A61Q 19/08; A61Q 19/00; A61Q 15/00; A61K 8/49; A61K 8/67

(52) U.S. Cl. CPC A61K 8/922; A61K 8/4946; A61K 8/97; A61K 8/891; A61K 8/365; A61K 8/678; A61K 8/673; A61K 8/44; A61Q 19/08; A61Q 19/00; A61Q 15/00; A61K 8/06; A61K 2800/592
USPC 424/65; 424/776; 424/744

(57) ABSTRACT

A composition for topical application to human skin having an effective amount of cosmetically suitable components comprising at least one nut oil, at least one thickener and/or emulsifier component and water. The at least one nut oil includes a substantial proportion of macadamia nut oil, and the composition includes only sufficient emulsifier to render the composition suitable for topical application. The composition is especially suitable for the treatment of aged persons, where it can improve their general health, and also assists to reduce undesirable body odour.

### TECHNICAL FIELD

The present invention relates to a cosmetic formulation suitable for topical application to human skin, which contains one or more nut, seed or plant oils, including a significant proportion of macadamia nut oil, as well as other components, including cosmetically suitable thickeners and emulsifiers. Preferably comparatively low levels of these thickeners and emulsifiers are present when compared to traditional products.

This composition is especially suitable for the treatment of aged persons, where it can improve their general health, and also helps to reduce undesirable body odour.

### BACKGROUND TO THE INVENTION

In aged individuals the loss of skin function produces dryness, inflammation, irritation and when mechanical or chemical impact occurs, also produces skin tears or other damage as well as general discomfort. This loss of skin barrier function results in a loss of quality of life and may reduce movement, increase the likelihood of infections, preclude restful sleep and impair general day to day functioning.

It has been found that specific oils containing palmitoleic acids and similar unsaturated glycerides, which are found in some plant species, such as in macadamia, function surprising well to maintain good skin physiology.

Recently, some published data also suggests the prolonged use of mineral oil based products, which are the basis for conventional skin care lotions and creams can compromise skin function, especially in the elderly. (See “Changes in skin barrier function following long-term treatment with moisturizers, a randomized controlled trial” by I. Buraczewska, B. Berne, M. Lindberg, H. Torma” and M. Loden). Many current skin treatments are primarily based on petroleum products, such as Vaseline®-based skin treatments, sorbolene, and even some baby oils.

It has also been discovered that applying a product having minimal emulsifier to the skin can reduce oil loss from subsequent water immersion such as with bathing. This allows the maintenance of skin having a high lipid content, so as to allow the restoration of healthy skin.

The unpleasant body odour that is particularly prevalent in aged persons is believed to result from the substance 2-nonenal that is generated from unsaturated fatty acids or lipid peroxides in skin surface lipids, such as palmitoleic acid (eg, see “2-Nonenal Newly Found in Human Body Odor Tends to Increase with Aging”, Haze et al, The Journal of Investigative Dermatology, Vol 116(4) pp 520-524, 4 Apr. 2001; & “Smelling Old?” in Science and Technology—Trends in Japan, 28 Sep. 1999, <http://web-japan.org/trends00/honbun/tj990921.html> and “The Smell of Age: Perception and Discrimination of Body Odors of Different Ages”, Mitro S, Gordon A R, Olsson M J, Lundström J N (2012) PLoS ONE 7(5): e38110. doi:10.1371/journal.pone.0038110.

A skin treatment formulation that reduces this odour, especially in aged persons, or at least does not enhance it occurring would be desirable.

It has now been found that skin creams that have a high level of nut oils, primarily of macadamia nut oil, will work very effectively as a skin conditioner and cosmetic treatment, especially for elderly or aged persons. It has also been found that such formulations act to minimise the unpleasant body odour that old persons sometimes exhibit.

Some skin treatment compositions utilising a macadamia component are known. For example, International Patent Publication no. WO 2010/091827 by Hubert Lengheimer (published 19 Aug. 2010) discloses a complex formulation that includes macadamia oil. However, the formulations described in this document disclose a cream having a minor portion of a lipophilic component that can itself contain a minor proportion of macadamia oil, leading to skin treatment creams that have, at most, 5% of macadamia oil. It has been found that effective skin creams should have a larger quantity of macadamia oil than this, to obtain the most desirable benefits.

In addition, International Patent Publication no. WO 2002/011693 by Provincia Italiana Della Congregazione Dei Figli Dell'immacolata Concezione—Istituto Dermopatico Dell'immacolata (published 14 Feb. 2002), discloses a sebum-mimicking mixture, which includes plant derived triglycerides, squalene, waxes, and cholesterol that is equivalent to the corresponding components in human sebum.

Some of these components are sourced from macadamia oil, and 35-70% of the preparation may consist of macadamia oil. However other components present, including squalene sourced from olive oil and jojoba wax, and cholesterol, are used to create a human sebum like mixture.

This sebum analogue mixture is not really suited for use with aged persons to minimise odour issues, as the replication of human sebum would not tend to help with the problem of body odour, because it would be providing more sebum for the chemical reactions that generate 2-nonenal.

In addition, this sebum analogue mixture is not really suited for use with aged persons in their usual environment, as the resulting mixture is generally a runny oil, and the skin lotions that are most suitable for use with aged persons should avoid potential problems with their use in locations and environments that aged persons inhabit. Therefore a skin treatment composition is desirable that has low levels of greasy components that otherwise would mark clothes and bed linen, and which is difficult to spill when used by persons having limited mobility so as to minimise safety issues of slippery floors, for example.

Accordingly, it would be useful to provide a solution that avoids or ameliorates any of the disadvantages present in the prior know skin lotions, or which provides another alternative to these prior art approaches.

### SUMMARY OF THE INVENTION

One aspect of the invention concerns a composition for topical application to human skin having an effective amount of cosmetically suitable components comprising (a) at least one nut oil; (b) at least one thickener and/or emulsifier component; and (c) water; characterised in that, component (a) includes a substantial proportion of macadamia nut oil, and the composition includes only sufficient emulsifier to render the composition suitable for topical application.

Another aspect of the invention relates to a method for improving the health of an aged person, which comprises topically applying to the skin of the aged person an effective amount of such a composition, at regular intervals over a significant time period.

Yet another aspect of the invention concerns a method for improving the body odour of an aged person, which comprises topically applying to the skin of the aged person an effective amount of this composition, at regular intervals over a significant time period. Preferably, this topical application is to a male aged person.

According to one aspect of the composition of the present invention, the at least one nut oil is selected from one or more of: macadamia nut oil or sea buckthorn seed oil. Preferably, the nut oil is substantially macadamia nut oil that is present in an amount of at least 25% by weight.

In one preferred embodiment, the composition also contains at least one skin soothing component, which is preferably one or more of macadamia nut wax, allantoin and/or its derivatives, and aloe vera and/or its derivatives.

Preferably, the thickener and/or emulsifier component is selected from one or more of: glycerol stearate, cetostearyl alcohol, PEG20 stearate, sodium polyacrylate, xanthan gum, potassium cetyl phosphate, lecithin, hydrogenated lecithin, beeswax, emulsifying wax, plant gums, plant exudates (gum ghatti, mastic).

In one preferred embodiment, the composition also comprises at least one film forming component, which is preferably dimethicone.

In one preferred embodiment, the composition also comprises at least one pH adjusting or buffering component, which is preferably citric acid.

In one preferred embodiment, the composition also comprises at least one preservative component, which is preferably one or more of: phenoxyethanol, ethylhexylglycerin, potassium sorbate and silver salts.

In one preferred embodiment, the composition also contains at least one vitamin or mineral component, which is preferably vitamin E and/or pantothenic acid (Vitamin B5).

In one preferred embodiment, the composition also contains at least one component that promotes blood circulation or blood vessel dilation, which is preferably L-arginine.

In one preferred embodiment, the components of the composition are present in the amounts of:

(a) one or more nut oils in an amount of from 25 to 75% by weight; at least one thickener and/or emulsifier component in an amount of from 0 to 20% by weight;

(c) and water present in an amount sufficient to render the cosmetic formulation spreadable on to skin; and

(d) one or more of: macadamia nut wax, allantoin and/or its derivatives, and aloe vera and/or its derivatives in an amount of from 1 to 10% by weight.

Preferably, the amount of water present is sufficient to prevent the composition from flowing freely and dripping during its transfer from a container on to skin of a patient during application of the composition.

In one particularly preferred embodiment, the components of the composition are present in the amounts of:

(a) one or more nut oils in an amount of from 20 to 30% by weight;

(b) at least one thickener and/or emulsifier component in an amount of from 3 to 10% by weight;

(c) and water present in an amount of 50 to 80% by weight; and

(d) one or more of macadamia nut wax, allantoin and/or its derivatives, and aloe vera and/or its derivatives in an amount of from 0.5 to 5% by weight.

Preferably, the amount of water present is about 65% by weight.

Most preferably, the composition contains substantially no mineral oils or squalene or squalene analogues.

### DETAILED DESCRIPTION OF PREFERRED EMBODIMENTS

The compositions of the present invention provide a topical skin care product comprising emulsified plant, nut or seed oil, including a substantial proportion of macadamia nut oil, and an aqueous phase, and some other cosmetically suitable ingredients for the required purpose.

This cosmetic formulation has utility especially for application to chronologically aged skin to prevent or minimise skin dryness, irritation and skin tears. The cosmetic formulation is particularly useful for aged skin that has been compromised. The other cosmetically acceptable ingredients that may also be included in the cosmetic formulation of the present invention can improve its functional, aesthetic and storage properties.

A skin care cosmetic product made in accordance with the present invention is particularly intended for the repair and maintenance of frail or age compromised skin. It may be readily absorbed into the skin when applied to the skin with gentle massage. It generally contains a minimal amount of emulsifier in order to produce a stable emulsion through homogenation. Ideally, the cosmetic formulation is essentially water miscible to allow for easy clean-up of spills, such that oily residues are not left behind.

The plant, nut or seed oil component is preferably macadamia oil, but may include other plant, nut or seed oils, particularly those with significant levels of palmitoleic acid such as Macadamia species and/or Sea Buckthorn (Hippophae rhamnoides) seed oil. Glyceride oils from other vegetable and algal sources such as triglyceride oils that have been refined by chemical processing such as fractionation, complexation, crystalisation, distillation, enzymic transformation, transesterification processes in a manner to increase the palmitoleic acid content above the natural plant levels may also be utilised by the invention.

However, the oil obtained from other nuts or seeds may also be included, such as that from almonds, cashews, and the like. Some additional examples include apricot kernel, arachis seed oil, amaranth oil, apricot kernel oil, arachis oil, avocado oil, baobab oil, black seed oil, vaccinium corymbosum (blueberry) seed oil, borage oil, brazil nut oil, camelina oil, camellia seed oil, cocoa butter, coconut oil, evening primrose oil, grapefruit seed oil, hazelnut oil, illipe butter, kukui nut oil, lime seed oil, linseed oil, macadamia oil, peach kernel oil, pink grapefruit seed oil, pomegranate seed oil, rosehip oil, sesame seed oil, shea nut butter, sweet almond oil, walnut oil, and wheat germ oil, for instance.

As used herein, the term “nut oil” is referring to what is generally and colloquially known as “nuts” and is not to be interpreted in a strictly botanical sense. The meaning of “nut” in the present document encompasses plant components that are not necessarily considered to be a nut in a strict botanical sense, but may also include seeds or other parts of plants. For example an almond is strictly a seed, and the macadamia nut is strictly a kernel, in a botanical sense, but both are considered to be a “nut” for the present invention, and as a source of the nut oil.

Preferably at least 15% of the formulation comprises the nut oil. More preferably the nut oil is present in an amount of from 25% to 50% by weight, and most preferably, is present in an amount of about 25% to 30%.

Macadamia oil is generally obtained by the cold pressing of the nuts (seeds) of the Macadamia temifolia tree followed by cooling and filtration to remove sediment and colour. Macadamia oil has advantages in use as a cosmetic oil due to its high emolliency and rapid skin penetration.

Dry (under active skin) and mature skin in the elderly is characterized as being thin and shiny, and can also have a scale like appearance. Dry skin can also be an issue for other persons, particularly as a result of smoking, poor diet, environmental exposure, prolonged sun exposure and some forms of post medical conditions, such as with chemotherapy, dialysis, diabetes, coeliacs disease, and so forth.

When such dry skin is pinched, its time to recover is noticeably prolonged. The skin's ability to retain moisture as a barrier is also reduced. This reduced barrier effectiveness manifests as inability to cope with external environmental pollutants such as soaps, clothing fibres, enzymes from clothes and bedding washing, and cosmetics. This leads to the skin being easily irritated by these factors.

The skin care preparations according to the present invention are ideally formulated to ameliorate the conditions of dry skin, in aged people, or with people who have damaged skin arising from medical or other conditions.

The skin care formulations of the present invention are generally prepared according to procedures used generally in the cosmetics industry. The preparation of a single phase mixture of oil and water for the purposes of preparing a suitably acceptable skin care product involves the emulsification of the desired oil and the water phase to give the desired oil/water particle size.

To ensure a product maintains an extended shelf life, one or more surfactants may be included as an emulsifier, so as to reduce the surface tension of the water and allow for small oil or water droplets to be formed, and to prevent their coalescence. Emulsifiers that may be used are generally selected from fatty acid glycerides, ethoxylated fatty acids, ethoxylated fatty esters of sugars such as sorbitol, ionic fatty acids and their salts such as stearic acid and triethanolamine stearate, phosphate esters of fatty acids and their ionic salts such as potassium cetyl phosphate, Teric, or Tween. A single emulsifier, or mixtures of several suitable ones may be used. In the cosmetic formulation of the present invention, the emulsifier component is preferably present in an amount of from 0 to 20%, and most preferably from 0 to 5%, by weight.

The presence of a substantial amount of macadamia oil in the product means that the amount of other substances that act as emulsifiers can be reduced when compared to conventional skin preparations.

Macadamia oil has a high level of palmitoleic acid (as tri-glycerides), in comparison to other nut-based oils, making it ideal to be easily emulsified. Consequently, formulations containing macadamia seed oil require less emulsifier than that traditionally used to formulate other such skin care products.

Alternatively, or in addition to including emulsifiers, mechanical energy or sheer may be utilised to achieve the desired particle size. Mechanical methods of emulsification can be employed to achieve this, including such methods as high pressure homogenation, or ultrasonic emulsification, such as Sonolation™. A partial or total absence of emulsifier components in the formulation is possible with mechanical methods of homogenation.

Additionally, this reduced amount of additional emulsifier component present in the formulation has the resulting and desirable effect of not soaping the oil away from the skin when exposed to water through bathing.

To also give the formulation of the present invention cosmetically acceptable “feel”, particularly to compromised skin, one or more thickener components, such as colloidal or gum materials may be added to increase the product viscosity. These allow the product to feel “substantial” when on the skin, and also operate chemically to improve the stability of the emulsion. Such materials may be xanthan gum, carrageenan gum, carbomer, polyacrylates, modified starches such as carboxymethyl cellulose, hydroxypropyl starch, and so on. In the cosmetic formulation of the present invention, preferably the thickener component is present in an amount of 0 to 10%, and most preferably as 1 to 2% by weight.

Skin soothing ingredients also may be included in the cosmetic formulation of the present invention, including herbal extracts and purified isolates of herbal materials. These may include, aloe vera (i.e., aloe vera leaf juice), comfry extract and the purified active constituent allantoin, or heather (caluna vulgaris) alcohol/water extract. Macadamia wax may also be utilised for this purpose. In the cosmetic formulation of the present invention, these skin soothing components may be present in an amount of from 1 to 10% by weight, and most preferably in an amount of 3% to 10%.

Antioxidant materials added to improve stability may also be included in the cosmetic formulation of the present invention, including butylated hydroxyl toluene, butylated hydroxy anisole, ascorbic acid, calcium ascorbate, erythorbic acid., sodium erythorbate, vitamin E, dl-alpha tocopherol, dl-alpha tocopherol acetate, tocopherols, rosemary officianalis extract, or rosemarinic acid. In the cosmetic formulation of the present invention, preferably the antioxidant component is present in an amount of 0 to 5%, and most preferably in an amount of 0 to 1% by weight.

As well, one or more film forming components may be included in the preparation, in order to form an occlusive barrier. A suitable such component may be dimethicone, or other cosmetically suitable silicone oils or polymerized siloxane compounds. Other examples of film forming components may include hydrogenated polyisobutene, poly vinyl pyrolidone, mineral oil, mineral wax, beeswax, lanolin, spermaceti wax, crospovidone, cellulose polymers such as hydroxypropylmethyl cellulose, hydroxyethyl cellulose, cocoa butter, jojoba oil, propylene glycols, and/or stearyl stearate, fruit wax such as orange peel wax, lemon peel wax, carnauba wax, polyglycerols such as polyglyceryl polyricinoleate, polyglycerol hydroxystearate. In the cosmetic formulation of the present invention, this component may be present in an amount of 0-15%, and most preferably as 1-2% by weight.

Preservatives may also be included in the cosmetic formulation of the invention, to improve the shelf life of the product. Such preservatives may be selected from those commonly used in the cosmetics industry. Examples of some suitable preservatives include potassium sorbate, or silver chloride. In the cosmetic formulation of the present invention, preferably, the minimum amount of preservative is included, generally in an amount of from 0-1% by weight.

It may also be useful to adjust the pH of the resulting formulation so that it matches (or at least closely resembles) the normal pH of skin, which is usually slightly acidic with adults, and somewhere between 4.5 and 6, and generally around 5.4. Some of the other components included in the formulation of the present invention may affect the formulation's overall pH, and so buffering agents or other cosmetically suitable materials may be included to allow for the desirable pH to be achieved. For example, an amount of citric acid, malic acid, lactic acid, tartaric acid or glycolic acid may be used for this purpose. When required, an amount of one or more of the selected acids is added to adjust the pH of the finished product to the desired pH. Generally a pH is chosen in the range of from 4.5 to 7.0, and ideally around 5.8.

One or more vitamins or minerals suitable in cosmetics, and especially those that provide nutritional and health benefits to skin, may also be included in the formulation. For example, vitamin E, or the vitamin B group vitamins, such as pantothenic acid (panthenol) for example may be added to the formulation of the present invention.

Additionally, ingredients that promote blood vessel dilation or improved blood flow may also be included in the composition. An example of such a component is the amino acid, L-arginine, or L-Citrulline for example. In the cosmetic formulation of the present invention, this component may be included in an amount of from 0 to 1%, and preferably from 0.001 to 0.1% by weight.

An appropriate amount of water may be used to make up an emulsion, and preferably the resulting formulation has a consistency so as to be pourable, but not too fast flowing that it would tend to drip. Ideally, the composition is provided in the form of a cream. This allows for the massage of the product into the skin. The mechanical action of massage can be an additional and important factor in improving the health of the skin. Alternatively the water may be derived from floral water condensates, vegetable or fruit juice extracts, floral extracts, mineral water sources, milk, milk processing by-products such as permeate, wine or fermented juice and homeopathic liquid preparations.

Especially when used in care facilities (and particularly aged care facilities), it is advantageous for the formulation to be difficult to spill, and with the least tendency for it to drip, when being applied on to elderly patients skin from a container. For these situations, it is preferred that the cosmetic formulations should be provided as a hard-to-spill lotion or cream. This may be achieved with a water content in the emulsion in the range of from around 60 to 70% and especially around 65%. For example, a cream having a viscosity of about 15,000 cps may be suitable for massage on to skin.

It is also useful, when the skin care formulation of the invention is being used with elderly people, who often may be bed ridden or who have limited mobility, for the formulations to minimise any grease, or staining when coming into contact with clothes or bedclothes. Therefore, the formulations are ideally made to include a low level of emulsifiers and thickeners, when compared with traditional skin care cosmetics. Having the base component of macadamia oil, which is a naturally occurring and vegetable origin substance, is advantageous in this regard, especially when compared with traditional skin care preparations that typically contain mineral oils.

Macadamia oil, being of natural plant origin, contains a complex mixture of components, and is thought to have anti-bacterial properties. Macadamia oil had a surprisingly high oxidative stability when compared with other plant oils. This oxidative stability seems to enhance the skin resilience. (e.g., see <http://alliance2u.com/pdf/Bulletin14.pdf>).

A skin care cream based on macadamia oil can have previously unsuspected properties, in that it appears able to minimise body odour. Elderly people, because their skin is somewhat compromised, have a tendency to develop body odour, which is thought to arise from bacterial colonisation of natural sebum that contains high proportions of dead skin cells. This is particularly a problem with men, who tend to produce more sebum than women. While an explanation for this effect arising from the present invention is unclear, the reduction in unpleasant body odour that results from using the formulations of the present invention may arise because of the special qualities of macadamia oil, and the comparatively low levels of emulsifier present in the formulations, in addition to the absence of mineral oils, when compared with traditional skin care lotions.

### EXAMPLES

### Example 1

### Formulation

A cosmetic preparation of the present invention for topical application was made with the following ingredients:

| MATERIAL NAME | % |
|---|---|
| POTASSIUM CETYL PHOSPHATE | 0.17% |
| PEG 20 STEARATE | 0.87% |
| MACADAMIA OIL | 25.00% |
| MACADAMIA WAX | 1.31% |
| GLYCERYL STEARATE | 2.62% |
| DIMETHICONE | 1.31% |
| DL ALPHA TOCOPHEROL ACETATE | 0.44% |
| CETYL ALCOHOL | 1.57% |
| WATER (PURIFIED) | 65.48% |
| PRESERVATIVE | 0.87% |
| XANTHAM GUM | 0.16% |
| ALOE VERA DRY 200:1 | 0.17% |
| CITRIC ACID | 0.13% |
| ALLANTOIN | 0.17% |
| SODIUM POLYCRYLATE | 0.17% |
| POTASSIUM SORBATE | 0.10% |

The formulation was prepared using conventional equipment, and by a process as used routinely in the cosmetic and pharmaceutical industries for preparing an oil-in-water emulsion. The emulsification proceeded with heating and cooling of the product in a controlled manner.

The final product was in the form of a cream, with a viscosity of 15,000 cps, which was suitable for massage on to skin.

### Example 2

### Trial

A formulation of the composition as prepared in accordance with Example 1 was trialed on human subjects to determine if it functioned to improve skin health, particularly of elderly persons.

Volunteer aged persons (20 women aged from 80 years or older) were divided randomly into two groups of 10. The participants of one group were treated with a macadamia-based cream according to the current invention, while the other group was treated with a conventional sorbolene-type cream. This sorbolene-type cream was manufactured using a standard and publically available formula, based on paraffin oil and glycerin.

Both products were white coloured creams of identical viscosity and texture, and packaged into identical looking containers, and the participants in the trial were not told which was which. Each container was marked with the participant's allocated number. Each container had a pump discharge, which provided a dose averaging 1.7 g, or slightly less than 2 ml.

In each subject the major areas treated were the lower arms, and/or lower legs on each side of the body. The average dosage during the trial period was about to one pump discharge for each area treated.

Using a scoring system as generally used in Admission Procedure for residents in Australian Aged Care Residential Homes, at the commencement of the trial, fifteen participants were assessed as “dry” and given the score of “1”. Also seventeen of the participants had their skin condition classified as “sensitive”, and given a score of “3”. All of the participants had additional skin conditions, which were included in the scoring, and their severity indicated by the magnitude of their individual skin integrity assessment scores. These included such descriptors as “tissue paper” skin (“1”), oedematous (“1”), clammy (“1”), discoloured (“2”), and broken/split (“3”). These scores were cumulative. These factors were assessed both before and after the trial, which was run over a twelve week period. The difference between them represents the change in skin condition attributable to the treatment given, and a reduction in the total score indicates an improvement in skin condition.

The results of the trial indicated that both the rate of improvement and the degree of improvement for the macadamia-based product was better than for the comparative sorbolene-type product, and this was measured at a statistically significant level.

The average skin integrity assessment score for the ten participants who used the sorbolene-type product changed from 7.1 at the beginning, to 4.4 at the conclusion of the trial. The average such score for the nine participants (one participant unfortunately died from unrelated causes during the trial period) using the macadamia-based cream changed from 6.7 at the start, to 2.6 at the conclusion of the trial.

Thus, the sorbolene-type cream gave an improvement of 2.7 integrity units, whereas the macadamia cream of the present invention had an improvement of 4.1 such units. The rate of improvement was also much quicker with macadamia-based cream.

After a “neutralising” gap of four weeks during which regular “home care” treatment was administered, a further trial was carried out using a modified “cross-over” methodology, whereby each of the groups were sub-divided into two sub-groups, and one of these subgroups was changed over to the other product, while the other subgroup remained using the original product.

Six participants originally on sorbolene-type product were switched to macadamia cream. Six participants originally on macadamia cream were switched to the sorbolene-type cream. Four persons being treated with sorbolene-type cream remained being treated as such, and three persons treated with macadamia-based cream remained with this treatment. Participants who had recorded no change on sorbolene treatments were switched, but other allocations were random. This trial period continued for twelve weeks.

The results from this second stage of the trial were significant. All participants who changed from sorbolene-type to macadamia-based creams recorded improved skin condition; one had the highest individual improvement score over all trial periods. The average improvement was +4.33 integrity units.

Notably, all but one of the participants who changed from macadamia-based cream to sorbolene-type creams recorded worsening skin condition. One actually regressed almost to the pre-trial “1” condition. The average regression was −1.0 integrity units. These results were statistically significant.

The people who remained on sorbolene-type cream were static, with a small bias towards worsening; the group average regression was −0.25 integrity units. Those people who remained on macadamia-based cream continued to record improvement; the group average improvement was +2.0 integrity units, which is statistically significant.

At the commencement of the trials the group's average skin integrity score was 6.8 units. Those participants receiving sorbolene-type treatment in either period averaged a decrease of 2.2 units over 12 weeks. Those receiving macadamia-based cream in either period averaged a decrease of 7.5 units over 12 weeks. This represents a more than 350% greater effect by use of the macadamia cream of the present invention. The results are statistically highly significant.

The improvement in skin health by using the macadamia-based cream according to the present invention was clearly demonstrated.

### INDUSTRIAL APPLICABILITY

The invention can be utilised in the cosmetics, pharmaceutical, medical and health care industries. The formulations in the present application are particularly of benefit for the management of aged skin, and of compromised skin prone to tears.

In this specification, unless the context clearly indicates otherwise, the term “comprising” has the non-exclusive meaning of the word, in the sense of “including at least” rather than the exclusive meaning in the sense of “consisting only of”. The same applies with corresponding grammatical changes to other forms of the word such as “comprise”, “comprises” and so on.

It will be apparent that obvious variations or modifications may be made which are in accordance with the spirit of the invention and which are intended to be part of the invention, and any such obvious variations or modifications are therefore within the scope of the invention.

1. A composition for topical application to human skin having an effective amount of cosmetically suitable components comprising:
a) macadamia oil in an amount of at least 25% by weight;
b) at least one emulsifier component and/or thickener in an amount of from 0 to 20% by weight; and
c) water in an amount of 50 to 80% by weight;
characterised in that the composition includes only sufficient emulsifier to render the composition suitable for topical application.

2. A composition of claim 1, which also contains at least one skin soothing component.

3. A composition of claim 2, wherein the at least one skin soothing component is selected from one or more of: macadamia nut wax, allantoin and/or its derivatives, and aloe vera and/or its derivatives.

4. A composition of claim 1, wherein the thickener and/or emulsifier component is selected from one or more of: glycerol stearate, cetostearyl alcohol, PEG20 stearate, sodium polyacrylate, xanthan gum, potassium cetyl phosphate, lecithin, hydrogenated lecithin, beeswax, emulsifying wax, plant gums or plant exudates, including gum ghatti and mastic.

5. A composition of claim 1, which also comprises at least one film forming component.

6. A composition of claim 5, wherein the film forming component is dimethicone.

7. A composition of claim 1, which also comprises at least one pH adjusting or buffering component.

8. A composition of claim 7, wherein the pH adjusting or buffering component is citric acid.

9. A composition of claim 1, which also comprises at least one preservative component.

10. A composition of claim 1, wherein the preservative component is selected from one or more of phenoxyethanol, ethylhexylglycerin, potassium sorbate and silver salts.

11. A composition of claim 1, which also comprises at least one vitamin or mineral component.

12. A composition of claim 11, wherein the vitamin or mineral component is vitamin E or pantothenic acid (Vitamin B5).

13. A composition of claim 1, which also comprises at least one component that promotes blood circulation or blood vessel dilation.

14. A composition of claim 13, wherein the at least one component that promotes blood circulation or blood vessel dilation is L-arginine.

15. A composition of claim 1, further comprising one or more of: macadamia nut wax, allantoin and/or its derivatives, and aloe vera and/or its derivatives in an amount of from 1 to 10% by weight.

16. A composition of claim 14, wherein the amount of water present is sufficient to prevent the composition from flowing freely and dripping during its transfer from a container on to skin of a patient during application of the composition.

17. A composition of claim 1, which contains substantially no mineral oils, squalene or squalene analogues.

18. A method for improving the health of an aged person, which comprises topically applying to the skin of the aged person an effective amount of the composition according to claim 1, at regular intervals over a significant time period.

19. A method for improving the body odour of an aged person, which comprises topically applying to the skin of the aged person an effective amount of the composition according to claim 1, at regular intervals over a significant time period.
